(19) United States
(12) Patent Application Publication
(10) Pub. No.: US 2026/0241161 A1
(43) Pub. Date: Aug. 20, 2026

(54) PACING CONDUCTOR

(71) Applicant: BOSTON SCIENTIFIC SCIMED, INC., MAPLE GROVE, MN (US)

(72) Inventor: Andrew John Mitterholzer, Eden Prairie, MN (US)

(73) Assignee: BOSTON SCIENTIFIC SCIMED, INC., MAPLE GROVE, MN (US)

(21) Appl. No.: 19/539,952

(22) Filed: Feb. 13, 2026

Related U.S. Application Data
(60) Provisional application No. 63/759,354, filed on Feb. 17, 2025.

(51) Int. Cl. A61N 1/05; A61B 18/14; A61N 1/362

(52) U.S. Cl. CPC A61N 1/056; A61B 18/1492; A61N 1/05; A61N 1/0573; A61N 1/3625; A61N 1/3627

(57) ABSTRACT

A conductive sleeve or film disposed on the outer surface of a delivery catheter to establish electrical connections in a pacing system during transcatheter procedures is disclosed. The conductive member may be configured as an integrated film or a sleeve slidably disposed onto the delivery catheter. The conductive sleeve or film serves to secure a negative terminal connection of a pacing system through direct contact with patient tissue.

### CROSS REFERENCE TO RELATED APPLICATIONS

This application claims the benefit of priority of U.S. Provisional Application No. 63/759,354 filed February 17, 2025, the entire disclosure of which is hereby incorporated by reference.

### TECHNICAL FIELD

The present disclosure generally relates to establishing electrical connections during transcatheter procedures. Particularly, but not exclusively, the present disclosure relates to strengthening electrical connections of a pacing system used during transcatheter procedures via a simplified circuit configuration.

### BACKGROUND

Transcatheter procedures often require establishing temporary electrical connections to provide cardiac pacing. Traditional approaches for establishing these connections typically require multiple vascular access sites and additional devices or users, which can increase procedural complexity and patient discomfort.

Current methods of establishing electrical connections during procedures like transcatheter aortic valve replacement (TAVR) can result in inconsistent electrical contact and potential signal loss. There remains a need for reliable solutions that can maintain consistent electrical contact with patient tissue while reducing procedural complexity and the number of vascular access sites required.

### BRIEF SUMMARY

This overview is intended to provide an introduction to the subject matter of the present patent application. It is not intended to provide an exclusive or exhaustive explanation. The detailed description is included to provide further information about the present patent application.

In an example, a medical apparatus includes a conductive member configured to be disposed on a proximal end region of a delivery catheter, where the conductive member includes either: a) a cylindrical segment of a conductive film configured to be integrated into an outer layer of the delivery catheter or b) a conductive sleeve including a flared proximal end configured to be slidably disposed over the delivery catheter. The apparatus includes a contact portion extending outward from the delivery catheter, where for configuration a), the contact portion is configured to be integrated into a proximal connector of the delivery catheter, and for configuration b), the contact portion includes an enlarged section on the conductive sleeve.

Alternatively or additionally to any of the examples above, the conductive member is disposed on the outer surface of the delivery catheter at the proximal end region of the delivery catheter.

Alternatively or additionally to any of the examples above, the conductive member extends distally from the proximal connector.

Alternatively or additionally to any of the examples above, the conductive member extends distally from the proximal connector for a length of at least 5 centimeters to establish direct electrical contact with the tissue at a location distal to an access site of the patient.

Alternatively or additionally to any of the examples above, the flared proximal end of the conductive sleeve has an outer diameter greater than an outer diameter of a hub of the proximal connector and configured to matingly engage the hub.

Alternatively or additionally to any of the examples above, the flared proximal end of the conductive sleeve is configured to abut a strain relief of a proximal connector.

Alternatively or additionally to any of the examples above, the conductive member, when the delivery catheter is in an operative position, is configured to establish contact with a tissue of a patient during advancement of the delivery catheter from the access site to the target treatment location.

Alternatively or additionally to any of the examples above, in the operative position, the conductive sleeve matingly engages the proximal connector.

Alternatively or additionally to any of the examples above, in the operative position, conductive sleeve maintains a fixed axial position relative to the delivery catheter.

Alternatively or additionally to any of the examples above, the conductive sleeve has a tapered distal end configured to provide an atraumatic profile.

Alternatively or additionally to any of the examples above, the conductive film is molded into the outer layer of the delivery catheter during formation of the catheter.

Alternatively or additionally to any of the examples above, the conductive film is thermally bonded to the outer layer of the delivery catheter.

Alternatively or additionally to any of the examples above, the conductive film is adhesively bonded to the outer layer of the delivery catheter.

Alternatively or additionally to any of the examples above, the conductive film is mechanically fastened to the delivery catheter.

Alternatively or additionally to any of the examples above, the conductive film extends circumferentially around the delivery catheter, where the conductive film terminates proximally at the proximal connector.

Alternatively or additionally to any of the examples above, the conductive member has a thickness from 2.5 micrometers to 250 micrometers.

Alternatively or additionally to any of the examples above, the conductive member has an outer diameter ranging from 0.5 millimeter to 9.95 millimeters.

Alternatively or additionally to any of the examples above, the conductive member has an inner diameter of 0.5 millimeters to 9.95 millimeters.

Alternatively or additionally to any of the examples above, the conductive member includes at least one of a metal, a metal alloy, and combinations thereof.

In an example, a pacing system includes a delivery catheter having a proximal end region and a distal end region, where the proximal end region includes a proximal connector. The system includes a conductive member disposed on an outer surface of the delivery catheter and extending distally from the proximal connector toward the distal end portion of the delivery catheter, where the conductive member is configured to establish electrical contact with tissue of a patient when the delivery catheter is advanced into the patient from an access site to a target treatment location. The system includes a contact portion radially outward from a proximal end region of the delivery catheter, where the contact portion is configured to establish an electrical connection with an electrical connector.

Alternatively or additionally to any of the examples above, the system further includes an external pulse generator having a negative terminal, where the contact portion is configured to electrically couple with the negative terminal via the electrical connector.

Alternatively or additionally to any of the examples above, the conductive member extends distally from the proximal connector for a length of at least 5 centimeters to establish direct electrical contact with the tissue at a location distal to an access site of the patient.

Alternatively or additionally to any of the examples above, the contact portion includes a textured surface configured to secure mechanical engagement and maintain electrical continuity with the conductive member.

Alternatively or additionally to any of the examples above, the conductive member has a thickness from 2.5 micrometers to 250 micrometers.

Alternatively or additionally to any of the examples above, the conductive member has an outer diameter ranging from 1 millimeter to 10 millimeters.

Alternatively or additionally to any of the examples above, the conductive member has an inner diameter of 0.5 millimeters to 9.95 millimeters.

Alternatively or additionally to any of the examples above, the conductive member includes at least one of a metal, a metal alloy, and combinations thereof.

Alternatively or additionally to any of the examples above, the conductive member includes a cylindrical segment of a conductive film integrated into an outer layer of the delivery catheter.

Alternatively or additionally to any of the examples above, the conductive film is molded into the outer layer of the delivery catheter during formation of the catheter.

Alternatively or additionally to any of the examples above, the conductive film is thermally bonded to the outer layer of the delivery catheter.

Alternatively or additionally to any of the examples above, the conductive film is adhesively bonded to the outer layer of the delivery catheter.

Alternatively or additionally to any of the examples above, the conductive film is mechanically fastened to the outer layer of the delivery catheter.

Alternatively or additionally to any of the examples above, the conductive film extends circumferentially around the delivery catheter, where the conductive film terminates proximally at the proximal connector.

Alternatively or additionally to any of the examples above, the conductive member includes a conductive sleeve slidably disposed over the delivery catheter, where the conductive sleeve configured to extend distally from the proximal connector when the pacing system is in an operative position.

Alternatively or additionally to any of the examples above, in the operative position, the conductive sleeve matingly engages the proximal connector and the conductive sleeve maintains a fixed axial position relative to the delivery catheter.

Alternatively or additionally to any of the examples above, the conductive sleeve includes a flared proximal end having an outer diameter greater than the proximal connector configured to matingly engage the proximal connector and maintain the conductive sleeve in a fixed axial position relative to the delivery catheter.

Alternatively or additionally to any of the examples above, the proximal connector further includes a cable hub and a strain relief.

Alternatively or additionally to any of the examples above, the conductive sleeve includes a flared proximal end having an outer diameter greater than an outer diameter of the hub of the proximal connector configured to matingly engage with the hub of the proximal connector to secure the conductive sleeve in a fixed axial position relative to the delivery catheter.

Alternatively or additionally to any of the examples above, the conductive sleeve abuts the strain relief of the proximal connector.

Alternatively or additionally to any of the examples above, the conductive sleeve has a tapered distal end configured to provide an atraumatic profile.

In an example, a method of establishing an electrical circuit during a transcatheter procedure includes advancing a delivery catheter through an access site to an operative position, where the delivery catheter includes a proximal end region having a conductive member, a proximal connector, and a contact portion. The method includes mechanically coupling a positive lead from a positive terminal of an external pulse generator to a guidewire, mechanically coupling a negative lead from a negative terminal of the external pulse generator to the contact portion of the delivery catheter using an electrical connector, where the conductive member is in contact with tissue of a patient when the delivery catheter is in an operative position, and delivering electrical stimuli from the positive terminal of the external pulse generator, where the electrical circuit is completed through the conductive member to the negative terminal of the external pulse generator.

Alternatively or additionally to any of the examples above, the conductive member includes a cylindrical segment of a conductive film integrated into an outer layer of the delivery catheter.

Alternatively or additionally to any of the examples above, the conductive member includes a conductive sleeve with a flared proximal end and a tapered distal end positioned over an exterior of the delivery catheter before insertion into the patient.

### BRIEF DESCRIPTION OF THE SEVERAL VIEWS OF THE DRAWINGS

FIG. 1 is a schematic diagram illustrating electrical connections of a pacing system during a transcatheter procedure.

FIG. 2A is a perspective view of a conductive member in accordance with one embodiment of the subject matter disclosed herein.

FIG. 2B is a side view of a pacing system including the conductive member of FIG. 2A.

FIG. 3A is side view of a conductive member in accordance with another embodiment of the subject matter disclosed herein.

FIG. 3B is a side view of a pacing system including the conductive member of FIG. 3A.

FIG. 3C is a perspective view of the conductive member of FIG. 3A.

FIG. 4 is a flow diagram in accordance with at least one embodiment of the subject matter disclosed herein.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

Direct wire pacing systems may be used during transcatheter procedures, such as transaortic valve replacement, to provide temporary pacing to the heart.

A conventional pacing system consists of an external pulse generator (herein referred to as “EPG”), a positive lead connected to a guidewire, and a negative lead connected to a patient. In conventional approaches for establishing a negative terminal connection, a negative lead extending from an external pulse generator is connected to the patient's abdominal tissue using a needle or alligator clip or by using a secondary tissue-contacting guidewire from a secondary access site. However, these configurations may be physically insecure and/or require multiple access points and/or users, increasing procedural complexity and potential complications.

In a conventional transaortic valve replacement, the delivery catheter is neither a component of the pacing system nor is it utilized in establishing the electrical connections needed for cardiac pacing. Rather, a delivery catheter serves as a mechanical conduit through which the guidewire is advanced from a vascular (i.e., femoral) access site, via a lumen in the delivery catheter, to reach the heart and deliver the replacement heart valve.

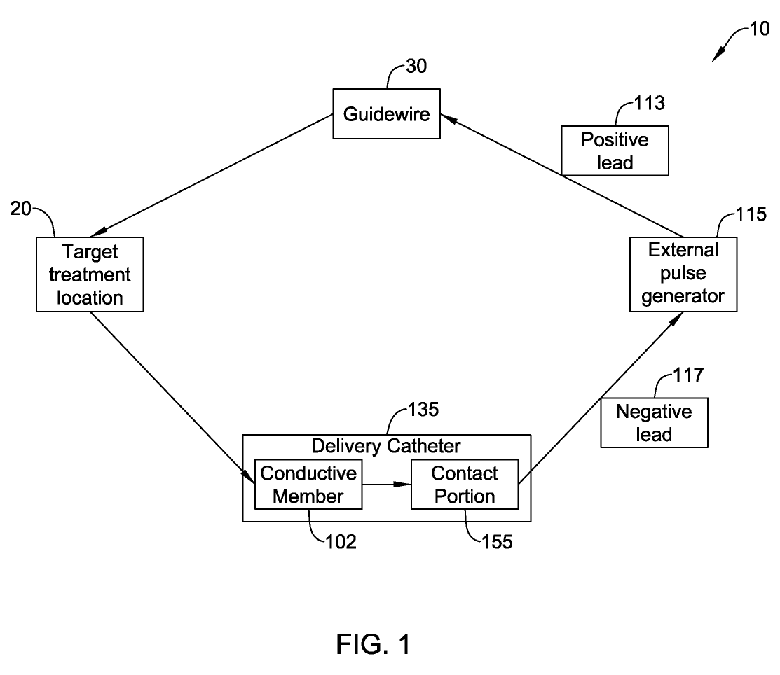

FIG. 1 illustrates exemplary electrical connections of a pacing system 10 during a transcatheter procedure. The transcatheter procedure may include inserting and navigating a guidewire 30 to a target treatment location 20 and inserting a delivery catheter 135 over the guidewire. An EPG 115 may be connected to the delivery catheter 135 via a negative lead 117 and to the guidewire 30 via a positive lead 113. Electrical stimulus begins at the positive terminal of the EPG 115 and flows through the positive lead 113 and the guidewire 30 at an access site to the target treatment location 20. In a TAVR procedure, the target treatment location 20 is heart vasculature, which may include the aortic valve annulus, the aortic root, ascending aorta, and the aortic arch. The access site for advancement of the delivery catheter may include, but is not limited to, a femoral artery access site, subclavian artery access site, carotid artery access site, apical access site, transaortic access site, transhepatic access site, and direct coronary sinus access site.

Once the stimulus is delivered to the target treatment location 20, the stimulus then returns to the negative terminal of the EPG 115 through the negative lead 117.

By including a conductive member 102 disposed on the delivery catheter 135, the disclosed pacing system provides a more secure electrical pathway. By disposing the conductive member 102 on the delivery catheter 135, the stimulus is in direct contact with the patient's body, better securing against inadvertent movements, reliance on insecure methods of attachment vulnerable to inadvertent dislodgement, and other sources of signal disruptions.

Rather than directly connecting the negative lead 117 to the circuit, the conductive member 102 may be disposed on the delivery catheter 135 and in direct contact with the arterial tissue. The conductive member 102 may be connected to a contact portion 155, then to the negative lead 117, and ultimately the negative terminal of EPG 115, completing the electrical circuit.

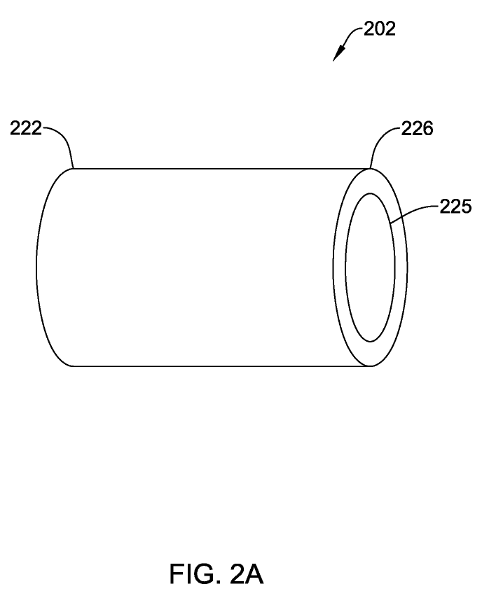

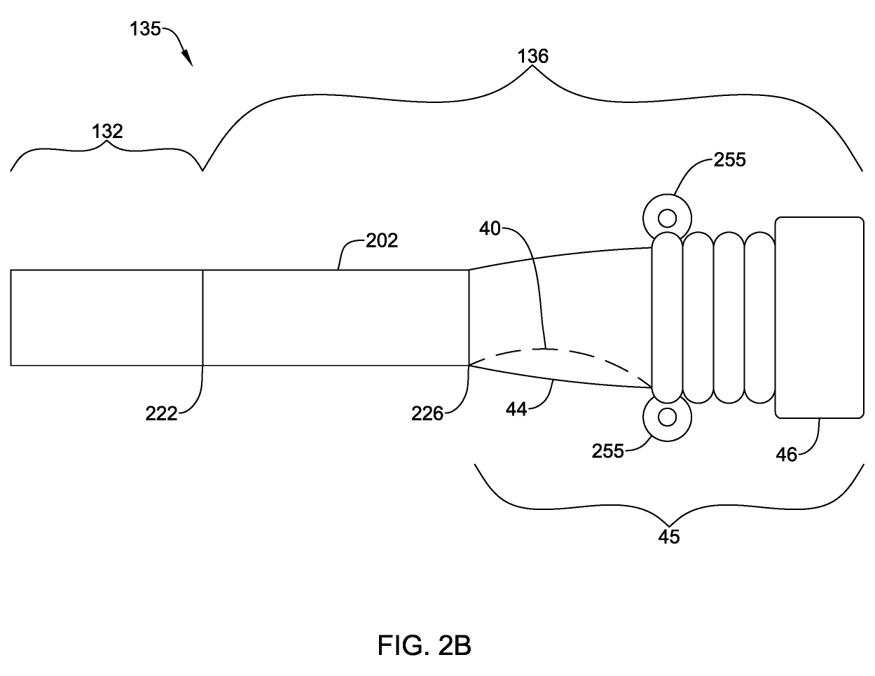

In some embodiments, as shown in FIG. 2A, the conductive member 102 may be a cylindrical segment of conductive film 202 having a distal end 222, a proximal end 226, and a lumen extending therebetween with an inner diameter 225. In some embodiments, the conductive film 202 may be integrated onto the outer layer of the delivery catheter.

The conductive film 202 may be manufactured of material that has electrical conductivity, such as a metal or a metal alloy. Examples of appropriate, but non-exclusive, materials for manufacture of the conductive film 202 include, but are not limited to, stainless steel, titanium, tantalum, tungsten, gold, platinum, palladium, and combinations thereof; and the metal alloy is selected from the group consisting of nickel-titanium alloys, nickel-chromium alloys, nickel-chromium-iron alloys, cobalt-chromium-nickel alloys, nickel-chromium-molybdenum alloys, nickel-molybdenum alloys, nickel-copper alloys, cobalt-chromium alloys, cobalt-chromium-molybdenum alloys, platinum-enriched stainless steel, and combinations and alloys thereof. Other examples include, but are not limited to a silver/silver chloride/carbon conductive coating, silver conductive ink, carbon-based conductive ink applied to substrates, such as polymer films (e.g., epoxy, silicone, polyethylene terephthalate (PET), and polymide base resins) and mylar films. Other considerations in the selection of the material may include, but are not limited to, strength, flexibility, corrosion resistance, and biocompatibility. Additionally, the manufacturing process may incorporate additional conductive elements, such as nanoparticles, to achieve the necessary electrical properties along the length of the conductive film 202 for establishing a secure negative terminal connection.

The conductive film 202 may range in radial thickness from 2.5 micrometers to 250 micrometers (9.8425x10⁻⁵ inches to 0.009842 inches). Thickness is driven to achieve mechanical strength for insertion and withdrawal, maintain electrical conductivity during manipulation and use, and to minimize size to avoid occluding the vascular access site or reducing the size of ancillary devices with which it is able to be used. In embodiments utilizing the polymer substrate, the polymer substrate may range in radial thickness from 75 micrometers to 150 micrometers (.00295 inches to .00591 inches).

FIG. 2B shows a side view of the delivery catheter 135 with the conductive film 202 integrated into the outer layer of the proximal end portion 136 of the delivery catheter 135. In some embodiments, the conductive film 202 may extend circumferentially around the outer surface of the delivery catheter 135. The circumferential configuration ensures consistent and direct tissue contact without impeding the ability of the delivery catheter 135 to be advanced through the vasculature from the access site to the target treatment location. In other embodiments, the conductive film 202 may be a discrete section of film fixed to the delivery catheter 135 that does not extend around the entire circumference of the delivery catheter 135.

In some embodiments, the conductive film 202 is molded into or thermally bonded to the outer layer of the delivery catheter 135 during its manufacture. In other embodiments, the conductive film 202 may be adhesively bonded to the outer layer of the delivery catheter 135. Other techniques of integration include lamination, overmolding, insert molding, and laser welding. In other embodiments, the conductive film 202 may be mechanically fastened to the delivery catheter. Methods of fastening may include fastening via clips, clamps, threaded connectors, rivets, pins, integrated mechanical interlocking features on the delivery catheter, molded inserts on the delivery catheter configured to receive the film. Any manner of integration allows the conductive film to maintain a continuous electrical pathway, while preserving the delivery catheter's ability to advance a treatment device through its internal lumen (not shown).

To ensure complete integration such that the conductive film 202 forms a continuous surface within the delivery catheter 135, the conductive film 202 may have an outer diameter equal with the outer diameter of delivery catheter 135. The outer diameter of the conductive film 202 may range from 1 millimeter to 10 millimeters (.03937 inches to .3937 inches). Briefly referring back to FIG. 2A, the conductive film 202 is illustrated with a constant inner diameter 225, however, the inner diameter of the conductive film 202 may vary along the length of the conductive film 202 in order to accommodate a delivery catheter 135 with a varying diameter in order to ensure complete and secure integration. In some embodiments, the inner diameter of the conductive film 202 ranges from 0.5 millimeters to 9.95millimeters (.0197 inches to 0.3917 inches). The conductive film 202 may have a radial thickness ranging from 0.25 millimeters to 2.5 millimeters (.009842 inches to .09842 inches).

The delivery catheter 135 includes a proximal connector 45 located at the proximal end portion 136 of the delivery catheter 135. See FIG. 2B. The proximal connector 45 serves to connect the internal conductive pathways of the pacing system and the external electrical connection to the negative lead (not illustrated). In some embodiments, and as illustrated, the proximal connector 45 includes a hub 44 and a strain relief 46. In some examples, and as illustrated in FIG. 2B, the conductive film 202 extends distally from the hub 44 of the proximal connector 45 towards the distal end portion 132 of the delivery catheter 135. The film extends distally to a point that, when inserted into the patient, ensures consistent direct contact with the patient tissue, with the conductive film 202 being completely contained inside the patient's body. In some embodiments, the distal end 222 of the conductive film 202 terminates at least 5 centimeters (1.97 inches) from the proximal connector 45 to ensure sufficient electrical contact and connection once the delivery catheter 135 is advanced into an operative position.

The proximal connector 45 may also include one or more contact portion 255. The contact portion 255 serves to provide secure anchoring of an electrical connector, and provides a mechanical coupling for attachment of the negative lead. Examples of electrical connectors include alligator clips, snap connectors, banana plugs, screw-type connectors, pin and socket connectors, spring loaded clamps, or other connectors made of conductive material.

In some embodiments, the contact portion 255 extends radially outward from the outermost surface of the proximal connector 45. As illustrated in FIG. 2B, the contact portion 255 is provided and integrated into the strain relief 46 of the proximal connector 45. In other embodiments, the contact portion 255 may be provided on the hub 44 of the proximal connector 45. A wire 40 may extend internally within the hub 44 to electrically connect the conductive film 202 to the contact portion 255.

In other embodiments, the contact portion 255 may be provided on the proximal end 226 of the conductive film 202, where the conductive film 202 is formed to extend radially outward from the outer surface of the delivery catheter 135, creating a gap between the outer surface of the delivery catheter and the film. This configuration allows the electrical connector to be inserted between the outer surface of the delivery catheter 135 and the conductive film 202. For example, in an embodiment wherein the electrical connector is an alligator clip, the alligator clip may pinch the conductive film 202 between the jaws of the alligator clip to establish the electrical connection.

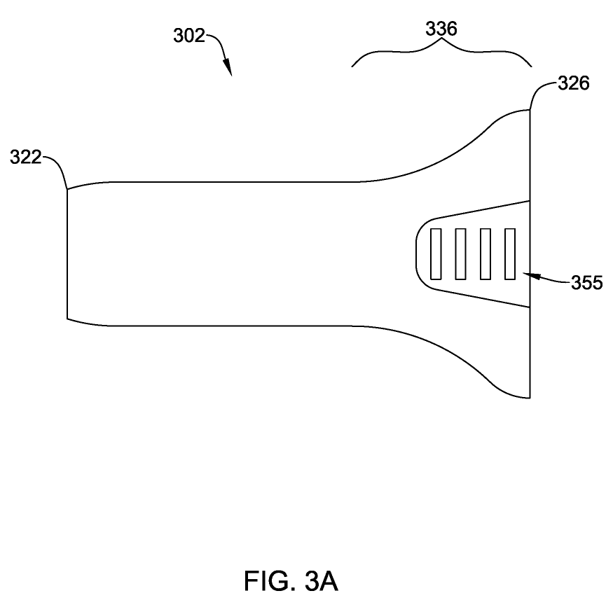

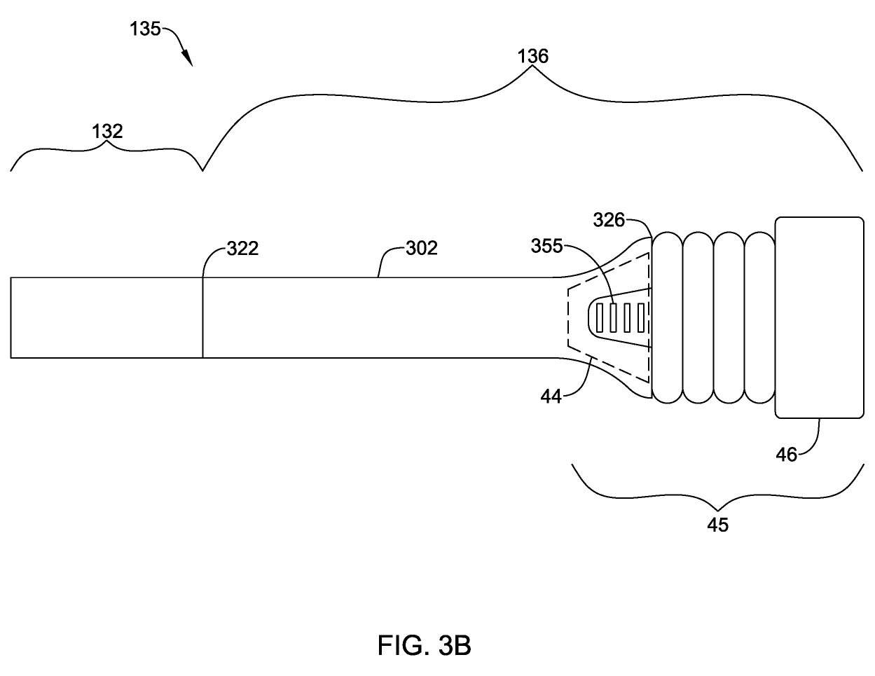

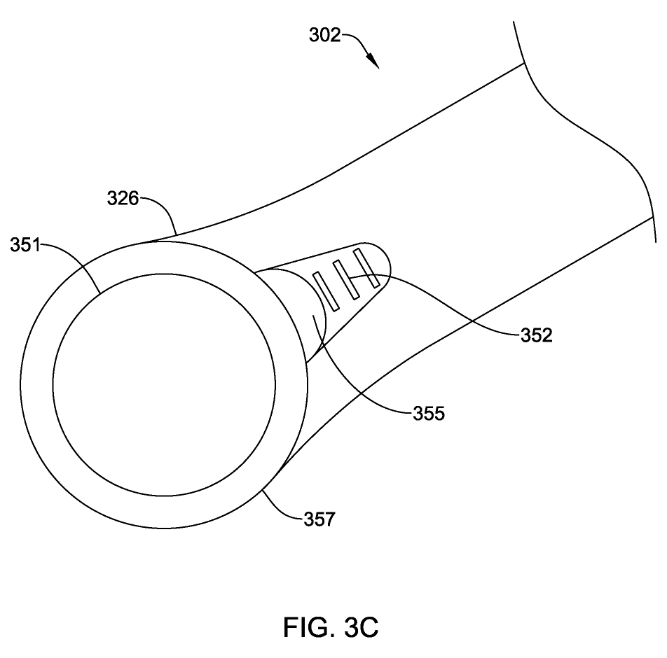

In some embodiments, as shown in FIG. 3A, the conductive member is a conductive sleeve 302 which is a separate piece that may be slidably disposed over the proximal end portion 136 of the delivery catheter 135. The conductive sleeve 302 may be separately manufactured from the other components of the pacing system. The materials appropriate for conductive sleeve 302 mirror the appropriate materials for the conductive film 202 shown in FIG. 2A and FIG. 2B, including: stainless steel; aluminum, tungsten; molybdenum, nickel and nickel alloys; zinc; cobalt-chromium alloys; titanium and titanium alloys; and precious metals such as platinum-iridium alloys, gold and gold alloys, silver and silver alloys, copper and copper alloys. Other examples include, but are not limited to a silver/silver chloride/carbon conductive coating, silver conductive ink, carbon-based conductive ink applied to substrates, such as polymer films (e.g., epoxy, silicone, polyethylene terephthalate (PET), and polymide base resins) and mylar films. Additionally, the manufacturing process may incorporate conductive elements, such as nanoparticles, to achieve the necessary electrical properties along the length of the conductive sleeve 302 for establishing the negative terminal connection.

As illustrated in FIG. 3A, the conductive sleeve 302 may include a flared proximal end 326 and a tapered distal end 322.The conductive sleeve 302 is configured to slide over the outer surface of the delivery catheter 135. Once the delivery catheter 135 is inserted into the conductive sleeve 302, the flared proximal end 326 of the conductive sleeve 302 is configured to matingly engage with the proximal connector 45. In this embodiment, the proximal connector functions as a mechanical interface point, ensuring that the flared proximal end 326 of the conductive sleeve 302 is secured into an operative position and maintains the electrical continuity of the pacing system.

The conductive sleeve 302 may also include a contact portion 355 configured to secure the electrical connector. In some embodiments, the contact portion 355 may extend distally from the proximal end 326 of conductive sleeve 302, as shown in FIG. 3A. In other embodiments, the contact portion 355 may be positioned at any point along the length of the proximal portion 336 of the conductive sleeve 302, ensuring that the contact portion 355 remains located outside the patient’s body to allow the user to utilize the electrical connector.

In FIG. 3B, the flared proximal end 326 of the conductive sleeve 302 is illustrated to matingly engage (i.e., extend over) the hub 44 of the proximal connector 45 and nesting against the strain relief 46 of the proximal connector 45. The conductive sleeve 302 may be sized and shaped to have a friction fit over the hub 44 and proximal end of the delivery catheter. The flared proximal end 326 may have an outer diameter greater than the outer diameter of the hub 44 or proximal connector 45 by a ratio of at least 10%. The design and capability of the conductive sleeve 302 to matingly engage with the proximal connector 45 enables the conductive sleeve 302 to maintain a fixed axial position relative to the delivery catheter 135 as the delivery catheter 135 is advanced from the patient access site through the vasculature to the target treatment location.

The outer diameter of the distal end 322 of the conductive sleeve 302 is such that once in the operative position, the conductive sleeve 302 forms a continuous outer surface with the distal end portion of delivery catheter 135. The tapered distal end 322 of the conductive sleeve 302 results in an atraumatic profile, reducing the likelihood of delivery catheter 135 snagging, encountering resistance, or otherwise interfering with the access site during advancement of the delivery catheter 135 into the patient.

FIG. 3C shows a perspective view of the conductive sleeve 302 from its proximal end 326. The outer surface 357 of the conductive sleeve 302 at the proximal end 326 is sized and configured such that once in the operative position, the outer surface 357 of the conductive sleeve 302 forms a continuous outer surface with the outer surface of the strain relief 46. The outer surface 357 of the conductive sleeve at its proximal end 326 may have a diameter range from 1 millimeter to 10 millimeters (.03937 inches to .3937 inches). To further ensure an atraumatic profile and secure the fixed axial position of the conductive sleeve 302, the inner surface 351 of the conductive sleeve 302 is sized to create a tight friction fit over the exterior surface of the delivery catheter. and the outer surface 357 of the conductive sleeve 302 at the distal end 322 of the conductive sleeve to form a continuous surface with the outer surface of delivery catheter 135. The outer surface 357 of the conductive sleeve at its distal end 322 may have a diameter range from 1 millimeter to 10 millimeters (.03937 inches to .3937 inches). The inner diameter of the conductive sleeve 302 may range from 0.5 to 9.95 millimeters (.0197 inches to 0.3917 inches).

The radial thickness of the conductive sleeve 302 may range between 2.5 micrometers to 250 micrometers (9.842x10⁻⁵ inches to 0.009842 inches). The conductive sleeve 302 may have a radial thickness configured to enable the user to position the conductive sleeve 302 over the delivery catheter 135 by sliding it over the proximal end of the delivery catheter 135. In some embodiments, the radial thickness of the proximal end 326 of the conductive sleeve 302 may have a larger radial thickness relative to its distal end, for example, to accommodate the contact portion 355.

In some embodiments, and as illustrated in FIG. 3C, the contact portion 355 extends radially outward from the outer surface 357 of the conductive sleeve 302. The radial extension allows for secure retention of the electrical connector. The contact portion 355 may also be enhanced with a textured surface to improve retention of the electrical connector and further ensure a secure negative terminal connection. Textured surfaces include, but are not limited to, ribbed, corrugated, or grooved (including helical, chevron, or knurled grooves). In the embodiment illustrated in FIG. 3C, the contact portion 355 includes a plurality of transverse ribs 352. For example, in some embodiments, the electrical connector comprises an alligator clip. Both jaws of the alligator clip may connect to the contact portion 355 by clipping the jaws onto one of the ribs 352 extending radially outward from the contact portion 355 of the conductive sleeve 302. In some embodiments, the contact portion 355 protrudes outward with a varying radial extension from the outer surface 357 of the conductive sleeve 302 to allow for attachment of the electrical connector. For example, in some embodiments, where the electrical connector comprises an alligator clip, the alligator clip may pinch to the contact portion 355 with one jaw facing the proximal end 326 of the conductive sleeve 302 and the other facing the distal end (not illustrated) of the conductive sleeve 302, ensuring a secure grip. The conductive sleeve 302 may be configured with an increased inner diameter 351 at the contact portion 355 to create a space between the conductive sleeve 302 and the delivery catheter specifically dimensioned to accommodate one jaw of the alligator clip, while allowing the other jaw to securely grip the outer surface of the contact portion 355. This configuration maintains the tight sliding fit elsewhere along the length of the conductive sleeve while providing a specific region for secure mechanical and electrical connection.

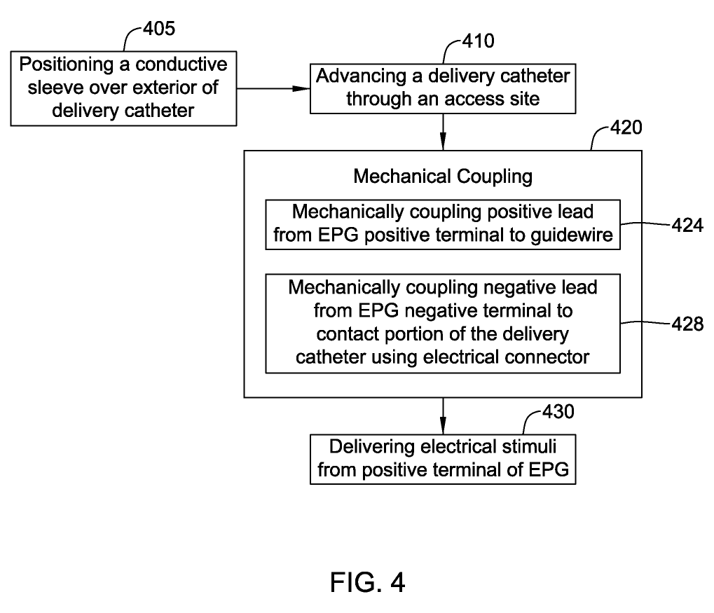

FIG. 4 shows an illustrative method of using the conductive members 202, 302 in the form of a flow diagram, typically performed by a user (e.g., clinician, physician or medical technician). More specifically, FIG. 4 shows a method of establishing the necessary electrical connections of the pacing system.

In embodiments where the conductive member is a separate conductive sleeve, such as the conductive sleeve 302 described above, the conductive sleeve is positioned 405 over the outer surface of the delivery catheter such that the flared proximal end of the conductive sleeve is matingly engaged with the proximal connector of the delivery catheter – either at the hub or the strain relief. In embodiments where the conductive member is a conductive film 202 integrated into the delivery catheter, no such action is necessary.

Once the conductive member has been positioned or integrated into the delivery catheter, the delivery catheter may be advanced 410 through an access site in the patient’s vasculature system toward the target treatment location. Potential access sites may include a femoral artery access site, subclavian artery access site, carotid artery access site, apical access site, transaortic access site, transhepatic access site, or a direct coronary sinus access site. Particularly for TAVR procedures, potential target treatment locations may include the aortic valve annulus, the aortic root, and ascending aorta, the aortic arch, or other heart vasculature.

After the conductive member is coupled to the delivery catheter and the distal end of the delivery catheter is positioned at the target treatment location, other components of the pacing system are mechanically coupled 420 to put the components of the pacing system in the operative position. The proximal end of the positive lead is mechanically coupled 424 to the positive terminal of the EPG and the distal end of the positive lead is mechanically coupled to the guidewire. The proximal end of the negative lead is mechanically coupled 428 to the negative terminal of the EPG and the distal end of the negative lead is mechanically coupled to the contact portion of the delivery catheter via an electrical connector. An electrical connector may include an alligator clip, a snap connector, a banana plug, a screw-type connector, a pin and socket connector, a spring loaded clamp, or other conductive connectors. Functionality of the pacing system is not impaired by the order of the mechanical coupling 420. Therefore, the positive lead may be coupled to the EPG and the guidewire first, followed by coupling the negative lead to the EPG and the contact portion of the delivery catheter, or the leads may be connected in the reverse order.

In either sequence, once mechanical coupling 420 of the positive lead 424 and the negative lead 428 are completed, the pacing system is ready to deliver electrical stimuli 430 from the EPG. As discussed in FIG. 1, once the EPG 115 is in operation, electrical stimuli begins at the positive terminal of the EPG, goes through the positive lead 113 and guidewire 30, is delivered to the target treatment location 20, and returns to the negative terminal of the EPG via the conductive member 102 disposed on the delivery catheter 135 and the negative lead 117.

It should be understood that this disclosure is, in many respects, only illustrative. Changes may be made in details, particularly in matters of shape and size, without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A medical apparatus, comprising:
a conductive member configured to be disposed on a proximal end region of a delivery catheter, wherein the conductive member comprises either:
a) a cylindrical segment of a conductive film configured to be integrated into an outer layer of the delivery catheter or
b) a conductive sleeve comprising a flared proximal end configured to be slidably disposed over the delivery catheter; and
a contact portion extending outward from the delivery catheter, wherein:
for configuration a), the contact portion is configured to be integrated into a proximal connector of the delivery catheter, and
for configuration b), the contact portion comprises an enlarged section on the conductive sleeve.

2. The medical apparatus of claim 1, wherein the conductive member is disposed on an outer surface of the delivery catheter at the proximal end region of the delivery catheter.

3. The medical apparatus of claim 1, wherein the conductive member extends distally from the proximal connector.

4. The medical apparatus of claim 3, wherein the conductive member extends distally from the proximal connector for a length of at least five (5) centimeters to establish direct electrical contact with tissue at a location distal to an access site of a patient.

5. The medical apparatus of claim 1, wherein the flared proximal end of the conductive sleeve has an outer diameter greater than an outer diameter of a hub of the proximal connector and configured to matingly engage the hub and maintain the conductive sleeve in a fixed axial position relative to the delivery catheter when the conductive sleeve is in an operative position.

6. The medical apparatus of claim 1, wherein the conductive sleeve has a tapered distal end configured to provide an atraumatic profile.

7. The medical apparatus of claim 1, wherein the conductive film is incorporated into the outer layer of the delivery catheter by molding during formation, thermal bonding, adhesive bonding, or mechanical fastening.

8. The medical apparatus of claim 1, wherein the conductive film extends circumferentially around the delivery catheter.

9. The medical apparatus of claim 1, wherein the conductive member has a radial thickness from 2.5 micrometers to 250 micrometers.

10. The medical apparatus of claim 1, wherein the conductive member has an outer diameter ranging from 1 millimeter to 10 millimeters.

11. The medical apparatus of claim 1, wherein the conductive member has an inner diameter of 0.5 millimeters to 9.95 millimeters.

12. The medical apparatus of claim 1, wherein the contact portion comprises a textured surface configured to secure mechanical engagement and maintain electrical continuity with the conductive member.

13. A pacing system, comprising:
a delivery catheter having a proximal end region and a distal end region, wherein the proximal end region comprises a proximal connector;
a conductive member disposed on an outer surface of the delivery catheter and extending distally from the proximal connector toward the distal end region of the delivery catheter, wherein the conductive member is configured to establish electrical contact with tissue of a patient when the delivery catheter is advanced into the patient from an access site to a target treatment location; and
a contact portion radially outward from the proximal end region of the delivery catheter, wherein the contact portion is configured to establish an electrical connection with an electrical connector.

14. The pacing system of claim 13, wherein the conductive member comprises a cylindrical segment of a conductive film integrated into an outer layer of the delivery catheter, wherein the contact portion is configured to electrically couple with a negative terminal of an external pulse generator via the electrical connector.

15. The pacing system of claim 13, wherein the conductive member comprises a conductive sleeve slidably disposed over the delivery catheter, wherein the conductive sleeve is configured to extend distally from the proximal connector when the pacing system is in an operative position and wherein the contact portion is configured to electrically couple with a negative terminal of an external pulse generator via the electrical connector.

16. The pacing system of claim 15, wherein, in the operative position, the conductive sleeve matingly engages the proximal connector and the conductive sleeve maintains a fixed axial position relative to the delivery catheter.

17. The pacing system of claim 15, wherein the conductive sleeve includes a flared proximal end having an outer diameter greater than the proximal connector configured to matingly engage the proximal connector and maintain the conductive sleeve in a fixed axial position relative to the delivery catheter.

18. A method of establishing an electrical circuit during a transcatheter procedure, comprising:
advancing a delivery catheter through an access site to an operative position,
wherein the delivery catheter includes a proximal end region having a conductive member, a proximal connector, and a contact portion;
mechanically coupling a positive lead from a positive terminal of an external pulse generator to a guidewire;
mechanically coupling a negative lead from a negative terminal of the external pulse generator to the contact portion of the delivery catheter using an electrical connector;
wherein the conductive member is in contact with tissue of a patient when the delivery catheter is in the operative position; and
delivering electrical stimuli from the positive terminal of the external pulse generator, wherein the electrical circuit is completed through the conductive member to the negative terminal of the external pulse generator.

19. The method of claim 18, wherein the conductive member comprises a cylindrical segment of a conductive film integrated into an outer layer of the delivery catheter.

20. The method of claim 18, wherein the conductive member comprises a conductive sleeve with a flared proximal end and a tapered distal end positioned over an exterior of the delivery catheter before insertion into the patient.
